(19) United States
(12) Patent Application Publication
(10) Pub. No.: US 2009/0022773 A1
(43) Pub. Date: Jan. 22, 2009

(54) POROUS AND BIOCOMPATIBLE CARRIER MATERIAL FOR TREATING BONE AND/OR CARTILAGE DEFECTS

(75) Inventors: Marco Springer, Esslingen (DE); Arne Briest, Karlsruhe (DE)

Correspondence Address: IP GROUP OF DLA PIPER US LLP, ONE LIBERTY PLACE, 1650 MARKET ST, SUITE 4900, PHILADELPHIA, PA, 19103, US

(73) Assignee: Ossacur AG, a corporation of Germany a corporation of Germany, Oberstenfeld (DE)

(21) Appl. No.: 12/095,342

(22) PCT Filed: Dec. 1, 2006
(86) PCT No.: PCT/EP06/11564

§ 371 Date: May 29, 2008

(30) Foreign Application Priority Data
Dec. 1, 2005 (DE) DE10 2005 058 507
May 31, 2006 (DE) DE10 2006 026 592

(51) Int. Cl. A61K 35/32; A61F 2/28; A61P 19/00

(52) U.S. Cl. 424/423; 424/548

(57) ABSTRACT

A porous and biocompatible carrier material for treating bone and/or cartilage defects, including a collagen of animal origin that has an active substance complex.

The invention relates to a porous and biocompatible carrier material, a method for its production, a kit, and the use of the carrier material for treating bone and/or cartilage defects.

The treatment of bone and cartilage defects represents one of the most important areas in intensive care medicine. For bone defects in particular, the number of treatment options has steadily increased in recent years, with interest increasingly focussing on biocompatible implants that have osteoinductive properties, i.e. that induce bone growth. For example, EP 0 500 556 B1 in particular has disclosed a very effective implant of cotton-like consistency composed of collagen, as skeleton structure, and in particular osteoinductive active substances.

However, in many cases, for example in the treatment of large bone defects, the implants require additional properties of dimensional stability. A very promising development in this connection is disclosed in EP 1 171 176 B1, which describes hollow bodies of various materials for restoring or stabilizing vertebral bodies or tubular bones, said hollow bodies comprising an active substance complex that contains collagen in particular.

Certain implantation sites in the body, however, are subject to particular pressure, which requires further dimensional stability of the implants.

The object of the invention is therefore to make available an implant which is further developed than the implants known from the prior art and which has excellent load-bearing properties and, in particular, excellent dimensional stability. The implant is also intended to promote osteoinduction (induction of bone growth) and/or chondrogenesis (induction of cartilage growth).

This object is achieved by a carrier material as described in claim 1. Preferred embodiments of this carrier material are set forth in dependent claims 2 through 12. Claims 13 through 16 concern a suitable method for producing such a carrier material. A corresponding kit is the subject matter of claims 17 and 18. Claim 19 concerns the use of the carrier material for treating, in particular for filling, bone and/or cartilage defects. The text of all the claims is hereby incorporated by reference into the content of the description.

According to the invention, the carrier material is a porous and biocompatible carrier material, in particular for treating bone and/or cartilage defects, the carrier material comprising a collagen of animal origin that has an active substance complex (collagenous active substance complex).

The invention makes available a porous and biocompatible carrier material which is suitable in particular for implantation in the area of bone and/or cartilage defects. The carrier material according to the invention comprises a collagenous active substance complex of animal origin. The carrier material itself preferably has load-bearing (osteoconductive) properties and, in particular, properties of dimensional stability, which make it especially suitable for treating, in particular for filling, bone and/or cartilage defects. In this way, it is possible for the carrier material according to the invention to be implanted in regions of the body which are subject to particular mechanical loads or which, for other reasons, place increased demands on the load-bearing function and, in particular, on the dimensional stability of an implant. By the choice of carrier material, and in particular by the way it is worked, the load-bearing properties of the carrier material according to the invention can be optimally adapted to the particular conditions of the implantation site; By means of the collagenous active substance complex, a particular advantage is that this promotes bone and/or cartilage growth in the area of the implantation site.

An active substance complex within the meaning of the present invention is to be understood as at least one active substance. For further features and details of the active substance or active substances, reference is made to the description that follows.

In a preferred embodiment, the collagen having an active substance complex is xenogenic, i.e. the collagen in question is a collegen of xenogenic origin. The collagen can in particular be of bovine, porcine or equine origin, particular preference being given to collagen of equine origin. The transmission of possible pathogens is particularly low in collagen of equine origin. Furthermore, the collagen of the carrier material according to the invention can also be human collagen.

The carrier material according to the invention preferably has pore sizes of between 100 and 400 μm, in particular of between 150 and 250 μm, preferably of ca. 200 μm. Such pore sizes, in particular those of the order of ca. 200 μm, are particularly preferred according to the invention, since they are especially suitable for the incorporation of blood vessels. In this way, it is possible to ensure a sufficient supply of oxygen and nutrients to the new bone and/or cartilage tissue forming inside and outside of the carrier material according to the invention.

The carrier material according to the invention can in particular have interconnecting pores, i.e. pores that are at least partially connected to one another via connecting channels. The interconnecting porosity of the carrier material according to the invention advantageously increases the (inner) surface area, which can be attributed in particular to the pores in the interior of the carrier material and to the connecting channels between the pores.

The collagenous active substance complex can be located on and/or in the carrier material. According to the invention, provision is made in particular for the surface of the carrier material, in particular the pores on the surface, to be coated at least partially, preferably completely, with the collagen having an active substance complex. If appropriate, the pores in the interior of the carrier material according to the invention, and in particular the inside walls of the connecting channels between the individual pores, are also preferably coated or, if appropriate, also filled at least partially, preferably completely, with the collagen having an active substance complex.

In some cases, it may be desirable that the pores, and in particular the connecting channels between the individual pores, are filled at least partially, preferably completely, with the collagen having an active substance complex.

In a preferred embodiment of the carrier material according to the invention, a collagenous active substance complex of bovine origin is the product sold commercially by the applicant under the name COLLOSS®. In a particularly preferred embodiment of the carrier material according to the invention, a collagenous active substance complex of equine origin is the product sold commercially by the applicant under the name COLLOSS® E.

In a further embodiment, the carrier material according to the invention comprises, in addition to the collagenous active substance complex, a metal or a metal alloy, preferably a light metal or a light metal alloy, particular preference being given to a light metal. The metal or the metal alloy can be present in particular in the form of a wire. The metal or the metal alloy can also be sintered. In this way, it is possible in particular to increase the dimensional stability of the carrier material according to the invention. According to the invention, the carrier material can be composed of a metal or a metal alloy and of the collagen having an active substance complex (collagenous active substance extract).

The carrier material according to the invention preferably comprises at least one metal from the group of magnesium, titanium vanadium and tantalum. The carrier material according to the invention is preferably composed of at least one metal from the aforementioned group and of the collagenous active substance complex. Magnesium, vanadium and tantalum are particularly preferred on account of their bioresorbability. Tantalum is also advantageously distinguished by being a metal that is easy to work, in particular to cut.

In another embodiment, the carrier material according to the invention comprises, in addition to the collagenous active substance complex, a ceramic, in particular a glass ceramic. The carrier material according to the invention is preferably composed of a ceramic and of the collagenous active substance complex.

In a particularly preferred embodiment, the carrier material according to the invention comprises, in addition to the collagenous active substance complex, phosphates, in particular alkali metal phosphates and/or alkaline earth metal phosphates. The phosphates are preferably calcium phosphate, in particular tricalcium phosphate, preferably β-tricalcium phosphate. Other preferred calcium phosphates are apatites, in particular hydroxyapatite. The β-tricalcium phosphate is preferably the product sold commercially by the applicant under the name OSSAPLAST®, a bioresorbable and granulated β-tricalcium phosphate, which is distinguished in particular by a high interconnecting porosity and in particular by a large specific surface area. The phosphates are preferably present as powders or granulates. The phosphates can have a grain size of between 200 and 3000 μm, in particular of between 800 and 2500 μm, preferably of between 1000 and 2000 μm. The carrier material according to the invention is preferably composed of phosphates, in particular alkali metal phosphates and/or alkaline earth metal phosphates, and of the collagen having an active substance complex.

In another embodiment, the carrier material according to the invention comprises, in addition to the collagen, at least one further polymer. This polymer can be resorbable or non-resorbable. This polymer can also be a copolymer or terpolymer. According to the invention, it is preferable that the carrier material comprises, in addition to the collagenous active substance complex, at least one polymer from the group of polyurethane, polylactic acid, polyglycolic acid, polyethylene, polypropylene and polytetrafluoroethylene. Polyurethane is preferred. The carrier material according to the invention is preferably composed of at least one polymer from the aforementioned group and of the collagenous active substance complex.

The carrier material according to the invention is preferably present as a lyophilisate, in particular as a co-lyophilisate. A co-lyophilisate is to be understood as a lyophilisate that contains at least two different materials. In addition to comprising the collagen with an active substance complex (collagenous active substance complex), the co-lyophilisate can in principle include any material with load-bearing properties, in particular with dimensional stability. This material is preferably at least one of the materials described in the preceding paragraphs for the carrier material according to the invention. The carrier material according to the invention is particularly preferably a co-lyophilisate based on the collagen that has an active substance and on a phosphate, in particular an alkali metal phosphate and/or alkaline earth metal phosphate. The carrier material according to the invention is preferably a co-lyophilisate of the collagen that has an active substance complex and of the phosphate, in particular the alkali metal phosphate and/or alkaline earth metal phosphate. For further properties of such phosphates, reference is made to the above description.

The carrier material according to the invention is expediently configured in the manner of an implant. The carrier material according to the invention can be present in particular as a planar textile structure, in particular as a knitted, braided, woven or nonwoven structure. The carrier material according to the invention is preferably present as a knitted structure. A knitted structure is particularly preferred on account of its elastic and in particular extensible properties.

The carrier material according to the invention can additionally be configured as a mesh, membrane, sponge or film. According to the invention, the porous and biocompatible carrier material is particularly configured as a sponge, preferably as a polyurethane sponge.

In another preferred embodiment, the carrier material according to the invention is present as a mesh, in particular as a hernia mesh. The filaments of the mesh are preferably encased by the collagen that has an active substance complex. According to the invention, it is also possible for the apertures of the mesh to be spanned at least partially, preferably completely, by the collagen that has an active substance complex, for example in the form of a film. The mesh can be present in particular as a knitted, woven or braided structure, a knitted mesh being preferred on account of its elastic and in particular extensible properties.

The carrier material according to the invention can also be present in the form of a metal sponge.

In another embodiment, the carrier material according to the invention is configured as a stent, in particular for spinal fusion.

In a preferred embodiment, the active substance complex, and the collagen having an active substance complex, are isolated, preferably extracted, from the same biological materials. The biological materials are in particular tissues and/or organs. The collagen and the active substance complex are preferably isolated from the same biological material, in particular from the same bone tissue. The collagen and the active substance complex can advantageously be obtained in a single isolation step, i.e. the collagen and the active substance complex are recovered simultaneously in one isolation step. If appropriate, the isolation step can also be repeated. In this way in particular, the active substance complex can be enriched.

The collagen having an active substance complex is preferably present as an extract. The collagen and the active substance complex are particularly preferably present as a common extract. According to the invention, the extract can in particular be composed of the collagen and of the active substance complex. The extract usually originates from biological materials, in particular from bone tissue. The extract can, in particular, be a lyophilisate. The nature of the extract, and in particular its production, will be dealt with in more detail below. The provision as an extract is particularly advantageous since in this way the active substance complex and the collagen can form a functional unit, the collagen serving as a direct skeleton and support structure for the active substances contained in the complex. The collagen furthermore serves as a stabilizing component and in particular prevents an at least partial loss of activity of the active substances contained in the complex. A common extract of active substance complex and collagen is preferably the lyophilized collagen extract of bovine origin sold commercially by the applicant under the name COLLOSS®. In a particularly preferred embodiment, a common extract of active substance complex and collagen is the lyophilized collagen extract of equine origin sold commercially by the applicant under the name COLLOSS® E.

In another embodiment, the collagen is present in native form, with single-helix collagen molecules (collagen fibrils) arranged in triple helices, which represent a kind of superstructure of natural collagen. Native collagen is particularly advantageous as a direct skelton and support structure for the active substance complex since, in the case of native collagen, the active substances contained in the complex are directly present in their “natural” environment.

Moreover, the collagen of the carrier material according to the invention can be a collagen that has been denatured, for example a collagen denatured by acid or base. Denatured collagen is composed at least partially of single-helix collagen molecules.

The collagen of the carrier material according to the invention is preferably present as renatured collagen, the single-helix collagen molecules being at least partially assembled. The renatured collagen can in particular be what is known as reconstituted collagen, which principally has the triple-helix superstructure of native collagen. A reconstituted collagen of this kind can be obtained, for example, by renaturation of collagen that has been denatured by acid or base.

In another embodiment of the carrier material according to the invention, the collagen, in particular the single-helix collagen molecules, is/are present at least partially, preferably completely, in a superstructure which is different than the triple-helix collagen configuration and has in particular a flat, preferably mesh-like configuration. This collagen superstructure permits improved coating and/or penetration of the carrier material which, because of its porosity, may have a non-uniform topology. The preparation of a renatured collagen of this kind, that has an active substance complex, is described in more detail below.

In another embodiment, the proportion of collagen with a superstructure differing from the triple-helix collagen configuration is at least 70 to 90%, in particular at least 80 to 90%, preferably at least ca. 90%, relative to the total amount of collagen in the carrier material according to the invention.

The lyophilized collagen extracts sold commercially by the applicant under the names COLLOSS® and COLLOSS® E are distinguished in particular by the fact that the collagen has a superstructure, in particular a flat superstrucure, preferably a mesh-like superstructure. For further details concerning COLLOSS® and COLLOSS® E, reference is made to the above description.

The carrier material according to the invention is also particularly advantageously distinguished by the fact that the active substances of the collagenous active substance complex are of native origin. This means that the active substances have their native biological activity and in particular originate from biological materials, preferably from bone tissue. The active substances of the active substance complex are preferably what are known as osteoinductive (inducing bone growth) and/or osteochondral (inducing cartilage growth) active substances. The active substances are preferably extracellular proteins and/or peptides. The active substances can in particular be recruiting, adhesion, growth and/or differentiation factors, with particular preference being given to growth and/or differentiation factors. The active substances are in particular what are known as BMPs (bovine morphogenetic proteins), in particular BMP-1 (bone mineralized protein 1) and BMP-2 (bone mineralized protein 2). The active substances of the collagenous active substance complex can also be FGF (fibroblast growth factor), PDGF (platelet-derived growth factor), TGF β1 (transforming growth factor β1) and VEGF (vascular endothelial growth factor). For further details concerning possible active substances, reference is made to EP 0 500 556 B1, the disclosure content of which is intended to be covered in this respect by the present invention. The lyophilized collagen extracts sold commercially by the applicant under the names COLLOSS® and COLLOSS® E are distinguished in particular by the fact that they at least partially comprise the active substances mentioned in this paragraph.

The collagen having an active substance complex is or can be preferably produced by:
reducing bone to powder,
demineralizing the powdered bone with an acid,
extracting the demineralized bone with a denaturing agent such that the collagen having an active substance complex is present as an extract,
removing the denaturing agent from the extract.

If appropriate, fat can be removed from the powdered bone with an organic solvent, in particular with a ketone and/or alcohol, either before or after the demineralization. Acetone in particular can be used as the ketone. The alcohol can be ethanol in particular. By the processes of demineralization and removal of fat, pathogens that are possibly contained in the animal bone material, and that are possibly transmissible, are effectively inactivated. The demineralization is carried out in particular with a mineral acid, for example hydrochloric acid, for example 0.6 mol hydrochloric acid.

Before the extraction step, the demineralized bone material is preferably subjected to incubation with chelating agents. The chelating agents are preferably EDTA (ethylene diamine tetraacetate) and/or TRIS (triethanolamine). In this way, divalent cations, for example magnesium and/or calcium ions, present in the bone tissue can be advantageously complexed.

The extraction of the demineralized bone material is preferably carried out with guanidine or a guanidine salt, for example guanidinium hydrochloride. In this way, a certain amount of collagen and in particular low molecular weight proteins and/or peptides, especially growth and/or differentiation factors, can be extracted from the bone tissue. The extraction step thus permits the simultaneous isolation and preparation of collagen and active substance complex in a single method step. If appropriate, the extraction step can in particular be repeated several times. Enrichment of the active substance complex in the extract can be achieved in this way. Enrichment of the active substance complex in the extract can also be achieved by adding further active substances to the collagenous active substance extract. These further active substances can be active substances that have been isolated or recovered in a separate method. Moreover, these further active substances can also be synthetic compounds.

According to the invention, the collagenous active substance complex present as an extract can undergo further purifying steps, for example dialysis steps. It is possible in this way to remove chaotropic compounds, in particular the strongly chaotropic salt guanidinium hydrochloride, and in particular to renature the collagen having an active substance complex.

It is also preferable, at the end of the extraction step, for the collagenous active substance complex present in the form of an extract to be subjected to shaping, in particular to lyophilization.

According to the invention, provision can in particular be made for the active substance complex to include at least one antimicrobial, in particular antibiotic, active substance. This active substance can in particular be added after production of the collagen having an active substance complex. Such a combination is preferably the product TARGOBONE® sold commercially by the applicant. TARGOBONE® is a collagen having an active substance complex, particularly in the manner of COLLOSS® or COLLOSS® E, and provided with the antibiotic teicoplanin.

Moreover, according to the invention, it may be advantageous for the active substance complex to be provided with cytostatics. In this way, possibly degenerated cells, in particular tumor cells, in the surrounding area of the implantation site can be destroyed The active substance complex can in particular also comprise recombinant active substances. The recombinant active substances are in particular active substances with properties that induce bone growth and/or induce cartilage growth.

The collagen of the carrier material according to the invention can be a collagen of type I, II, III or IV, particular preference being given to collagen of type I. The collagen can also be tendon and/or ligament collagen, preferably tendon collagen. The collagen can be recombinant.

In another embodiment of the carrier material according to the invention, the collagen having an active substance complex can be present in the form of a mixture, in particular lyophilized, together with pure collagen (free of active substances), in particular with collagen of type I, preferably with tendon collagen of type I. In this way, the osteoconductive, i.e. load-bearing, properties of the carrier material according to the invention can be additionally improved. The collagen having an active substance complex and the pure collagen are preferably present as a common lyophilisate.

In another embodiment, the carrier material according to the invention comprises physiological fluids. Possible physiological fluids are in particular body fluids, preferably blood. The carrier material according to the invention preferably comprises in particular coagulated blood. The blood can in particular be patient's blood, preferably homologous patient's blood, i.e. the patient's own blood. The carrier material according to the invention can further comprise a blood fraction, in particular a PRP (platelet rich plasma) fraction. The constituents present in the blood or in blood fractions, in particular blood platelets and preferably their released substances, can themselves have osteoinductive and/or osteochondral properties.

The carrier material according to the invention can also be of a putty-like nature. The putty-like nature can in particular be obtained by addition of physiological fluids to the carrier material. For example, the carrier material can be converted into a putty-like carrier material by addition of blood. The putty-like properties of the carrier material according to the invention permit in a particularly advantageous manner an adaptation of the carrier material to different defect topologies.

In another embodiment, the carrier material according to the invention is sterilized or present in sterilized form. Sterilization can be performed using the customary methods known to persons skilled in the art, particular preference being given to sterilization by radioactive irradiation, in particular by gamma radiation, and/or ethylene oxide gas sterilization. The carrier material according to the invention can also be treated aseptically and/or produced in an aseptic state, in particular by using sterile-filtered antibiotic solutions, for example a sterile-filtered gentamicin solution. A treatment of the carrier material with antibiotics is particularly preferred, since in this way it is possible to avoid undesired impairment of the active substances contained in the active substance complex, in particular growth and/or differentiation factors.

The carrier material according to the invention is preferably present in a packaged form. Possible packages are in particular syringes or blister packs. Thermoformed plastic parts, as are described in the examples, are also particularly suitable.

The present invention also relates to a method for producing a porous and biocompatible carrier material, comprising the following steps:
coating and/or penetration of the carrier material with a collagen of animal origin that has an active substance complex and, if appropriate, is present in a dispersing agent, and, if appropriate,
removal of the dispersing agent from the coated and/or penetrated carrier material.

The collagen having an active substance complex (collagenous active substance complex) is preferably transferred into a dispersing agent before the coating and/or penetration of the carrier material, in order to form a dispersion. Depending on the choice of dispersing agent, the coating and/or penetration is carried out from a solution or a suspension, preferably from a suspension. The coating and/or penetration of the carrier material is preferably carried out with a suspension. In the case of the suspension, it is in particular an aqueous suspension. The aqueous suspension is preferably obtained from a phsyiological saline solution and the collagenous active substance complex. If appropriate, the dispersing agent can be at least partially removed before the coating and/or penetration of the carrier material, in order to concentrate the dispersed collagen. The dispersing agent can be removed, for example, by application of a vacuum, in particular during a centrifugation step.

The coating and/or penetration of the carrier material according to the invention can be carried out in particular by an immersion method, in which the carrier material to be coated and/or penetrated is immersed in a solution or suspension, preferably in a suspension, of the collagenous active substance complex. In the case of swellable carrier materials, the coating and/or penetration can be carried out in particular by swelling the carrier material in a solution or suspension of the collagen having an active substance complex.

In another embodiment of the method according to the invention, the collagenous active substance complex is used in solid form, in particular in lyophilized form, for the coating and/or penetration of the carrier material. The carrier material can in particular be mixed together with the collagen having an active substance complex. According to the invention, provision can also be made for a dispersing agent to be added to the obtained mixture of carrier material and collagenous active substance complex. The addition of a dispersing agent, for example a physiological saline solution, facilitates in particular the coating and/or penetration of the carrier material with the collagenous active substance complex.

In a preferred embodiment of the method according to the invention, the coated and/or penetrated carrier material is incubated with a physiological fluid, in particular with a body fluid, preferably with blood. As has already been mentioned, the blood can be patient's blood, preferably homologous patient's blood. The carrier material is usually incubated at room temperature. The carrier material is preferably incubated in a temperature range of between 20° C. and 40° C., in particular at ca. 25° C. or ca. 37° C. The incubation itself is expediently carried out in an oven. In the case of blood, the incubation is usually carried out until the blood is preferably completely coagulated.

The removal of the dispersing agent can be carried out by application of a vacuum. The removal of the dispersing agent is preferably carried out by freeze-drying (lyophilization). In this way, the collagen having an active substance complex is advantageously built up, in particular in its planar and preferably mesh-like superstructure, on the surface of and/or inside of the carrier material (inner pores and in particular connecting channels between the individual pores).

The coating and/or penetration steps and, if appropriate, the removal of the dispersing agent can in particular be repeated several times in order to increase the amount of the collagen having an active substance complex on and/or in the carrier material.

The method according to the invention can be carried out using all the containers known to a person skilled in the art and in particular in use in medical laboratories. The method according to the invention can be carried out, for example, in syringes or dishes, preferably in syringes.

Regarding the collagenous active substance complex used for the coating and/or penetration of the carrier material, reference is made to the above description.

The present invention further relates to a kit for treating, in particular for filling, bone and/or cartilage defects, the kit comprising at least a porous and biocompatible carrier material and a collagen of animal origin that has an active substance complex (collagenous active substance complex). In a particular embodiment of the kit, the porous and biocompatible carrier material and the collagen having an active substance complex are spatially separate from each other. According to the invention, provision can be made for the carrier material to be coated and/or penetrated with the collagen having an active substance complex just before implantation. The kit according to the invention can in particular include suitable containers for storage, and in particular for intermediate storage, of the porous and biocompatible carrier material and of the collagen of animal origin having an active substance complex, for example vials with rolled rims, syringes, boats. For further details concerning the kit, in particular concerning the porous and biocompatible carrier material and the collagen having an active substance complex, reference is made to the above description.

The present invention finally relates to the use of the porous and biocompatible carrier material, comprising a collagen of animal orgin having an active substance complex (collagenous active substance complex), for treating, in particular for filling, bone and/or cartilage defects. The porous and biocompatible carrier material according to the present invention is preferably suitable for treating, in particular for filling, spinal column deformations, preferably spinal fusions. For further details and features, reference is made to the above description.

Further features and details of the invention will become clear from the following description of preferred embodiments in the form of examples. Here, the individual features can each be realized singly or in several combinations with one another. The examples serve only to explain the present invention, which is not in any way restricted to them.

### EXAMPLE 1

### Doping of a Polyurethane Foam with COLLOSS®

20 mg of COLLOSS® lyophilisate (product from the applicant, its properties having been explained in greater detail in the description) are placed with the aid of pincers into a 5 ml syringe. Thereafter, ca. 0.5 ml of physiological saline solution is introduced into the syringe. After an incubation time of ca. 5 minutes, and if appropriate after moving the syringe plunger back and forth several times (for better mixing), the obtained viscous COLLOSS® suspension is pressed out into a suitable vessel, for example a boat. A polyurethane foam (diameter 7 mm, height 3 mm) is immersed in the COLLOSS® suspension. During immersion in the viscous COLLOSS® suspension, the polyurethane foam can, if appropriate, be squeezed together several times, for example with pincers. After removal of the physiological saline solution by freeze-drying, a polyurethane foam doped with COLLOSS® is obtained.

### EXAMPLE 2

### Doping of a Polyurethane Foam with COLLOSS® E

20 mg of COLLOSS® E lyophilisate (product from the applicant, its properties having been explained in greater detail in the description) are placed with the aid of pincers into a 5 ml syringe. Thereafter, ca. 0.5 ml of physiological saline solution is introduced into the syringe. After an incubation time of ca. 5 minutes, and if appropriate after moving the syringe plunger back and forth several times (for better mixing), the obtained viscous COLLOSS® E suspension is pressed out into a suitable vessel, for example a boat. A polyurethane foam (diameter 7 mm, height 3 mm) is immersed in the COLLOSS® E suspension. During immersion in the viscous COLLOSS® E suspension, the polyurethane foam can, if appropriate, be squeezed together several times, for example with pincers. After removal of the physiological saline solution by freeze-drying, a polyurethane foam doped with COLLOSS® E is obtained.

### EXAMPLE 3

### Production of a Putty-like Material from COLLOSS® E and OSSAPLAST®

A pellet of COLLOSS® E (product from the applicant, its properties having been explained in greater detail in the description) is mixed homogeneously with granulate spheres of OSSAPLAST® (product from the applicant, its properties having been explained in greater detail in the description). The mixture is then introduced into a syringe and subjected to lyophilization. Thereafter, autologous patient's blood is added to the lyophilisate present in the syringe. The incubation with the patient's blood is carried out in an oven at a temperature of ca. 37° C. until complete coagulation of the blood. If appropriate, the mixture of autologous patient's blood and lyophilisate in the syringe is compressed by actuation of the syringe plunger. After coagulation of the blood, the obtained mixture is pressed out of the syringe. Alternatively, the obtained mixture can also be removed from the syringe by opening the syringe head.

### EXAMPLE 4

### Production of a Putty-like Material from COLLOSS® E and OSSAPLAST®

A lyophilisate of COLLOSS® E (product from the applicant, its properties having been explained in greater detail in the description) is mixed homogeneously with granulate spheres of OSSAPLAST® (product from the applicant, its properties having been explained in greater detail in the description) and then transferred into a syringe. Autologous patient's blood is added to the mixture present in the syringe. The incubation of the mixture is carried out in an oven at a temperature of ca. 37° C. until coagulation of the blood. The obtained mixture is then pressed out of the syringe. Alternatively, the obtained mixture can also be removed from the syringe by opening the syringe head.

The products prepared according to Examples 3 and 4 have a putty-like consistency. The use of OSSAPLAST® for producing the products leads to a marked increase in the osteoconductive or load-bearing properties and to an increased surface area of the product. The formulation of the products with autologous patient's blood permits in principle an improved and in particular successful treatment of bone and/or cartilage defects.

### EXAMPLE 5

### Production of a Co-lyophilisate of COLLOSS® E and OSSAPLAST®

Starting out from an aqueous suspension of COLLOSS® E (product from the applicant, its properties having been explained in greater detail in the description), the dry matter of COLLOSS® E is determined. Thereafter, the suspension is concentrated by ultracentrifugation (9,800 rpm, Via Suprafuge 22) for 2 hours. The supernatant is then transferred to a sterile glass vial and stored in the cool. From the concentrated COLLOSS® E suspension, the dry matter is determined or set to ca. 12±1 mg per gram of suspension. The concentrated suspension is then introduced into syringes or blisters. The dosing is effected by gravimetry. A corresponding gravimetrically determined quantity of OSSAPLAST® (product from the applicant, its properties having been explained in greater detail in the description) in a grain size of between 1000 and 2000 μm is then added to an enriched COLLOSS® E suspension. The volume of the mixture is recorded, and the mixture is homogenized with a spatula to a dough-like composition. Thereafter, the homogenized mixture of COLLOSS® E and OSSAPLAST® is subjected to lyophilization, in particular to joint lyophilization. The volume of the lyophilisate is then recorded. The lyophilisate is a mechanically stable, sponge-like product.

Alternatively, and preferably, the obtained dough-like mixture of COLLOSS® E and OSSAPLAST® is introduced into a thermoformed plastic serving as a package and is lyophilized therein. The package may then, if appropriate, be closed (sterile), and the mechanically stable, sponge-like product is obtained therein, its shape and dimensions being defined by the shape and dimensions of the package.

1-19. (canceled)

20. A porous and biocompatible carrier material for treating bone and/or cartilage defects, comprising a collagen of animal origin that has an active substance complex.

21. The carrier material as claimed in claim 20, wherein the collagen is xenogenic.

22. The carrier material as claimed in claim 20, wherein the carrier material has pore sizes of between about 100 and about 400 μm.

23. The carrier material as claimed in claim 20, wherein the carrier material further comprises a metal or a metal alloy.

24. The carrier material as claimed in claim 23, wherein the metal or metal alloy is a light metal or a light metal alloy.

25. The carrier material as claimed in claim 20, wherein the carrier material comprises at least one metal selected from the group consisting of magnesium, titanium, vanadium and tantalum.

26. The carrier material as claimed in claim 20, wherein the carrier material further comprises a ceramic.

27. The carrier material as claimed in claim 20, wherein the carrier material further comprises phosphates.

28. The carrier material as claimed in claim 27, wherein phosphates are alkali metal phosphates and/or alkaline earth metal phosphates.

29. The carrier material as claimed in claim 27, wherein the phosphates are calcium phosphate.

30. The carrier material as claimed in claim 29, wherein the phosphates are tricalcium phosphate.

31. The carrier material as claimed in claim 20, wherein the carrier material further comprises at least one polymer.

32. The carrier material as claimed in claim 31, wherein the polymer is at least one selected from the group consisting of polyurethane, polylactic acid, polyglycolic acid, polyethylene, polypropylene and polytetrafluoroethylene.

33. The carrier material as claimed in claim 20, wherein the carrier material is present as a lyophilistate.

34. The carrier material as claimed in claim 33, wherein the lyophilisate is a co-lyophilisate.

35. The carrier material as claimed in claim 20, wherein the collagen having an active substance complex is present as an extract, the active substance complex and the collagen being present as a common extract.

36. The carrier material as claimed in claim 20, wherein the active substance complex contains active substances.

37. The carrier material as claimed in claim 36, wherein the active substances are osteoinductive and/or osteochondral active substances.

38. A method for producing a porous and biocompatible carrier material as claimed in claim 20, comprising:
coating and/or penetrating the carrier material with a collagen of animal origin that has an active substance complex;
optionally adding a dispersing agent to the carrier material; and
removing the dispersing agent from the coated and/or penetrated carrier material.

39. The method as claimed in claim 38, wherein the coating and/or penetration is carried out from a solution or a suspension.

40. The method as claimed in claim 38, wherein the coated and/or penetrated carrier material is incubated with a physiological fluid.

41. The method as claimed in claim 38, wherein removing the dispersion agent is carried out by freeze-drying.

42. A kit for treating bone and/or cartilage defects, comprising:
a porous and biocompatible carrier material; and
a collagen of animal origin that has an active substance complex.

43. A method of treating bone and/or cartilage defects comprising applying a therapeutically effective amount of the carrier material as claimed in claim 20 to bone and/or cartilage.
